(19) United States
(12) Patent Application Publication
(10) Pub. No.: US 2008/0167450 A1
(43) Pub. Date: Jul. 10, 2008

(54) Methods of purifying proteins

(76) Inventor: Hai Pan, Camarillo, CA (US)

Correspondence Address: AMGEN INC., MAIL STOP 28-2-C, ONE AMGEN CENTER DRIVE, THOUSAND OAKS, CA, 91320-1799, US

(21) Appl. No.: 12/008,160

(22) Filed: Jan. 7, 2008

Related U.S. Application Data
(60) Provisional application No. 60/878,727, filed on Jan. 5, 2007.

(51) Int. Cl. C07K 1/14

(52) U.S. Cl. 530/387.3; 530/387.1

(57) ABSTRACT

The present invention relates to methods of purifying and analyzing preparations of Fc domain containing polypeptides comprising binding said polypeptide to protein A, more particularly to a protein A column, and eluting with a pH gradient elution system. This method enhances separation of aggregates, multimers and modified versions of the protein from the single Fc domain containing polypeptide.

This application claims the benefit of U.S. Provisional Application No. 60/878,727, filed Jan. 5, 2007, which is hereby incorporated by reference.

### FIELD OF THE INVENTION

The present invention relates to methods of purifying and analyzing preparations of Fc domain containing polypeptides comprising binding said polypeptide to protein A, more particularly to a protein A column, and eluting with a pH gradient elution system. This method enhances separation of aggregates, multimers and modified versions of the protein from the single Fc domain containing polypeptide.

### BACKGROUND OF THE INVENTION

Many antibody therapies require high doses over a long period of time, which requires large amounts of purified product per patient. However, purification of proteins, particularly recombinantly expressed proteins, to a non-aggregated, correctly folded, purified preparation is a difficult, time consuming process and potentially expensive. Thus, manufacturing systems to supply purified antibody to meet the demand for antibody therapeutics is a challenge.

Purification of proteins typically involves several steps that can include selective precipitation, immunoaffinity, and various types of chromatography among other steps. It is also common that more than one purification step is necessary in order to isolate a protein in highly pure form. Even robust purification processes can yield a heterogeneous mixture of protein that contains a small percentage of clipped, misfolded, insoluble, and/or aggregated forms of the protein. Furthermore, when a correctly folded protein is purified to a very high degree the final product may also have other modifications such as oxidation and variable glycosylation patterns in side chains. Thus, obtaining a pure protein can be challenging, particularly in the pharmaceutical industry seeking to manufacture consistent and safe therapeutic proteins.

Protein purification has traditionally been based on different properties, for example, specific affinity to a substrate (e.g., immunoprecipitation, protein A), size, charge and solubility, between a desired protein to be purified and contaminant proteins, such as those extra proteins from a host cell. For Fc domain containing proteins it is common to use protein A column purification as a column. However, since proteins tend to aggregate, and the aggregates still retain Fc domains as well as other properties of the native molecule, they will often co-purify from these columns.

Various methods have been developed to remove modified forms of proteins and aggregate forms including using size exclusion chromatography (SEC), which removes larger molecules preferentially, but this method is limited in it's loading capacity. Ion exchange chromatography has also been used as well as hydrophobic interaction chromatography (U.S. Pat. Nos. 6,620,918 and 5,429,746). However, each of these methods requires additional columns and material for purification of material that has been applied to a protein A column and thus are disadvantaged in cost and time.

The Fc portion of the IgG serves to bind various effector molecules of the immune system as well as molecules that determine pharmacokinetics and pharmacodynamics of the antibody. Fc is also known to interact with FcRn, which regulates IgG concentrations both in serum and through the body. The receptor weakly binds with Fc domain at neutral pH, but shows increased affinity at pH 6. The FcRn interaction site on IgG has been mapped using a combination of X-ray crystallography, functional studies and site-directed mutagenesis and is also near the protein A interaction domain. It has been shown that modification in Fc potentially can alter these important functions for antibody therapeutics. Thus, removal of undesirable oxidized protein species is desired.

Accordingly there is a need in the art for improved purification methods and for methods for analyzing protein preparations of Fc domain containing proteins.

### SUMMARY OF THE INVENTION

The invention contemplates binding an Fc domain containing polypeptide to protein A, and in a particular embodiment to a protein A column, at or near neutral pH and eluting the bound molecule using a pH gradient, thereby separating the desired product from multimers and/or aggregates of the protein. This separation method can be used in a manufacturing process as a polishing step to increase the homogeneity of the protein of interest, and can also be used as an analytical tool to better understand the product quality at various stages of the manufacturing process. In a particular embodiment, the elution range starts near pH 7.0 and does not fall below pH 3.0. In another embodiment, the Fc domain containing polypeptide is an antibody.

The invention also finds utility in detecting and quantifying the amount of oxidation of residues in an Fc domain containing protein. In a particular embodiment, the method is useful for identifying and quantifying the amount of oxidized methionine found in an Fc domain containing protein.

Additional features and variations of the invention will be apparent to those skilled in the art from the entirety of this application, and all such features are intended as aspects of the invention.

### BRIEF DESCRIPTION OF THE DRAWINGS

FIG. 1 depicts the typical chromatogram of a crude cell culture sample of an IgG2 antibody under bind and release mode.

FIG. 2 depicts the typical chromatogram of a crude cell culture sample of an IgG2 antibody under pH gradient elution.

FIG. 3 depicts the size exclusion chromatograms of main and post-peak fractions collected from pH gradient Protein A chromatography.

FIG. 4 depicts the DSC analysis for main peak, pre-peak and fully oxidized species.

FIG. 5 depicts the pH gradient Protein A chromatography for various antibodies.

FIG. 6 depicts the pH gradient Protein A chromatography for a Fc fusion protein.

FIG. 7 depicts the measured pH during the pH gradient Protein A chromatography.

### DETAILED DESCRIPTION

The present inventors discovered that eluting Fc domain containing polypeptides that are bound to protein A in a pH gradient can sequentially elute various forms of the protein, such as unmodified monomers, modified monomers, multimers and aggregates from the column. This process results in an increased separation of the proteins and elution fractions containing more homogeneous proteins relative to conventional methods using step elution.

One aspect of the invention utilizes a buffer, where any buffer that provides buffering activity in the pH range from 7 to 3 is suitable to separate the aggregates and Fc modified form from normal Fc containing polypeptide. While tris/acetate buffers are used in the working examples below, other buffers will readily be recognized as suitable for use according to the invention.

An aggregate, as used herein, is understood to include any multimer of an Fc domain containing protein including fragments. Thus, in the example of an antibody a monomer is considered to be the normal structure of two heavy and two light chains, whereas an aggregate is this normal structure that has associated with additional other antibodies. In the case of a Fc domain fusion protein, a monomer is considered to be the usual dimer of Fc domain fused to a sequence such as a peptide or receptor domain, for example. Thus, a typical aggregate is a multimer of the monomeric species.

As used herein, it is understood that an Fc domain containing polypeptide can be either an antibody or fragment thereof, or an Fc domain fused to a heterologous sequence or sequences.

The term “antibody” is used in the broadest sense and includes fully assembled antibodies, monoclonal antibodies, polyclonal antibodies, multispecific antibodies (e.g., bispecific antibodies) and recombinant peptides comprising the forgoing as long as they exhibit the desired biological activity. Multimers or aggregates of intact molecules and/or fragments, including chemically derivatized antibodies, are contemplated. Antibodies of any isotype class or subclass, including IgG, IgM, IgD, IgA, and IgE, IgG1, IgG2, IgG3, IgG4, IgA1 and IgA2, are contemplated. Different isotypes have different effector functions; for example, IgG1 and IgG3 isotypes have antibody-dependent cellular cytotoxicity (ADCC) activity.

The term “monoclonal antibody” as used herein refers to an antibody obtained from a population of substantially homogeneous antibodies, i.e., the individual antibodies comprising the population are identical except for possible naturally occurring mutations or alternative post-translational modifications that may be present in minor amounts, whether produced from hybridomas or recombinant DNA techniques. Nonlimiting examples of monoclonal antibodies include murine, chimeric, humanized, or human antibodies, or variants or derivatives thereof. Humanizing or modifying antibody sequence to be more human-like is described in, e.g., Jones et al., Nature 321:522 525 (1986); Morrison et al., Proc. Natl. Acad. Sci., U.S.A., 81:6851 6855 (1984); Morrison and Oi, Adv. Immunol., 44:65 92 (1988); Verhoeyer et al., Science 239:1534 1536 (1988); Padlan, Molec. Immun. 28:489 498 (1991); Padlan, Molec. Immunol. 31(3):169 217 (1994); and Kettleborough, C. A. et al., Protein Eng. 4(7):773 83 (1991); Co, M. S., et al. (1994), J. Immunol. 152, 2968-2976); Studnicka et al. Protein Engineering 7: 805-814 (1994); each of which is incorporated herein by reference. One method for isolating human monoclonal antibodies is the use of phage display technology. Phage display is described in e.g., Dower et al., WO 91/17271, McCafferty et al., WO 92/01047, and Caton and Koprowski, Proc. Natl. Acad. Sci. USA, 87:6450-6454 (1990), each of which is incorporated herein by reference.

Another method for isolating human monoclonal antibodies uses transgenic animals that have no endogenous immunoglobulin production and are engineered to contain human immunoglobulin loci. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); WO 91/10741, WO 96/34096, WO 98/24893, or U.S. patent application publication nos. 20030194404, 20030031667 or 20020199213; each incorporated herein by reference.

The term “variant” when used in connection with antibodies refers to polypeptide sequence of an antibody that contains at least one amino acid substitution, deletion, or insertion in the variable region or the portion equivalent to the variable region, provided that the variant retains the desired binding affinity or biological activity. In addition, the antibodies of the invention may have amino acid modifications in the constant region to modify effector function of the antibody, including half-life or clearance, ADCC and/or CDC activity. Such modifications can enhance pharmacokinetics or enhance the effectiveness of the antibody in treating cancer, for example. See Shields et al., J. Biol. Chem., 276(9):6591-6604 (2001), incorporated by reference herein in its entirety. In the case of IgG1, modifications to the constant region, particularly the hinge or CH2 region, may increase or decrease effector function, including ADCC and/or CDC activity. In other embodiments, an IgG2 constant region is modified to decrease antibody-antigen aggregate formation. In the case of IgG4, modifications to the constant region, particularly the hinge region, may reduce the formation of half-antibodies.

The term “derivative” when used in connection with antibodies refers to antibodies covalently modified by conjugation to therapeutic or diagnostic agents, labeling (e.g., with radionuclides or various enzymes), covalent polymer attachment such as pegylation (derivatization with polyethylene glycol) and insertion or substitution by chemical synthesis of non-natural amino acids. Derivatives of the invention will retain the binding properties of underivatized molecules of the invention. Conjugation of cancer-targeting antibodies to cytotoxic agent, for example, radioactive isotopes (e.g., I131, I125, Y90 and Re186), chemotherapeutic agents, or toxins, may enhance destruction of cancerous cells.

### Modification of Polypeptides

Any combination of deletion, insertion, and substitution is made to arrive at the final construct, provided that the final construct possesses the desired characteristics. The polypeptides or antibodies of the invention can be modified by techniques well-known to one of ordinary skill in the art. Potential mutations include insertion, deletion or substitution of one or more residues. Insertions or deletions are preferably in the range of about 1 to 5 amino acids, more preferably 1 to 3, and most preferably 1 or 2 amino acids. The variation may be introduced by systematically making substitutions of amino acids in an antibody using recombinant techniques well known in the art and assaying the resulting recombinant variants for activity. Nucleic acid alterations can be made at sites that differ in the nucleic acids from different species (variable positions) or in highly conserved regions (constant regions). Methods for altering antibody sequences and expressing antibody polypeptide compositions useful in the invention are described in greater detail below.

Substitution refers to a modified polypeptide with at least one amino acid residue in the polypeptide molecule removed and a different residue inserted in its place. Substitution includes substitution with alanine, a conservative substitution, or a non-conservative substitution. Conservative substitutions involve replacing an amino acid with another member of its class. Non-conservative substitutions involve replacing a member of one of these classes with a member of another class. Substitutional mutagenesis within any of the surface exposed regions of a polypeptide, such as the hypervariable or CDR regions or framework regions of an antibody, is contemplated. Further substitutions include, in the case of an antibody, replacement with a corresponding amino acid residue at the same position from a different IgG subclass (e.g. replacing an IgG1 residue with a corresponding IgG2 residue at that position).

Conservative amino acid substitutions are made on the basis of similarity in polarity, charge, solubility, hydrophobicity, hydrophilicity, and/or the amphipathic nature of the residues involved. For example, nonpolar (hydrophobic) amino acids include alanine (Ala, A), leucine (Leu, L), isoleucine (Ile, I), valine (Val, V), proline (Pro, P), phenylalanine (Phe, F), tryptophan (Trp, W), and methionine (Met, M); polar neutral amino acids include glycine (Gly, G), serine (Ser, S), threonine (Thr, T), cysteine (Cys, C), tyrosine (Tyr, Y), asparagine (Asn, N), and glutamine (Gln, Q); positively charged (basic) amino acids include arginine (Arg, R), lysine (Lys, K), and histidine (His, H); and negatively charged (acidic) amino acids include aspartic acid (Asp, D) and glutamic acid (Glu, E).

Techniques for cloning and expressing nucleotide and polypeptide sequences are well-established in the art [see e.g. Sambrook et al., Molecular Cloning: A Laboratory Manual, 2nd Edition, Cold Spring Harbor, N. Y. (1989)]. For example, the nucleic acid encoding a polypeptide or a modified polypeptide is isolated and inserted into a replicable vector for further cloning (amplification of the DNA) or for expression. DNA encoding the monoclonal antibody is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more selective marker genes, an enhancer element, a promoter, and a transcription termination sequence.

Methods for making bispecific or other multispecific antibodies are known in the art and include chemical cross-linking, use of leucine zippers [Kostelny et al., J. Immunol. 148:1547-1553, 1992]; scFv dimers [Gruber et al., J. Immunol. 152: 5368, 1994], linear antibodies [Zapata et al., Protein Eng. 8:1057-62, 1995]; and chelating recombinant antibodies [Neri et al., J Mol Biol. 246:367-73, 1995].

Accordingly, the purification methods of the invention can be used to purify antibodies, human antibodies, humanized antibodies, chimeric antibodies, i.e. antibodies having human constant antibody immunoglobulin domains coupled to one or more murine variable antibody immunoglobulin domain, and/or non-human antibodies, or fragments thereof. Exemplary antibodies are Herceptin® (Trastuzumab), a recombinant DNA-derived humanized monoclonal antibody that selectively binds to the extracellular domain of the human epidermal growth factor receptor 2 (Her2) proto-oncogene; and Rituxan® (Rituximab), a genetically engineered chimeric murine/human monoclonal antibody directed against the CD20 antigen found on the surface of normal and malignant B lymphocytes. Other exemplary antibodies include Avastin® (bevacizumab), Bexxar® (Tositumomab), Campath® (Alemtuzumab), Erbitux® (Cetuximab), Humira® (Adalimumab), Raptiva® (efalizumab), Remicade® (Infliximab), ReoPro® (Abciximab), Simulect® (Basiliximab), Synagis® (Palivizumab), Xolair® (Omalizumab), Zenapax® (Daclizumab), Zevalin® (Ibritumomab Tiuxetan), or Mylotarg® (gemtuzumab ozogamicin), Vectibix® (panitumumab). Exemplary Fc fusion proteins include fusion to soluble forms of receptors, enzymes, variants, derivatives, or analogs.

Examples of antibodies or antibody/cytotoxin or antibody/luminophore conjugates contemplated for use in the invention include those that recognize one or more of the following antigens: CD2, CD3, CD4, CD8, CD11a, CD14, CD18, CD20, CD22, CD23, CD25, CD33, CD40, CD44, CD52, CD80 (B7.1), CD86 (B7.2), CD147, IL-4, IL-5, IL-8, IL-10, IL-2 receptor, IL-4 receptor, IL-6 receptor, IL-13 receptor, PDGF-β, VEGF, TGF, TGF-β2, TGF-β1, EGF receptor, VEGF receptor, C5 complement, IgE, tumor antigen CA125, tumor antigen MUC1, PEM antigen, LCG (which is a gene product that is expressed in association with lung cancer), HER-2, a tumor-associated glycoprotein TAG-72, the SK-1 antigen, tumor-associated epitopes that are present in elevated levels in the sera of patients with colon and/or pancreatic cancer, cancer-associated epitopes or polypeptides expressed on breast, colon, squamous cell, prostate, pancreatic, lung, and/or kidney cancer cells and/or on melanoma, glioma, or neuroblastoma cells, TRAIL receptors 1, 2, 3 and 4, the necrotic core of a tumor, integrin alpha 4 beta 7, the integrin VLA-4, B2 integrins, TNF-α, the adhesion molecule VAP-1, epithelial cell adhesion molecule (EpCAM), intercellular adhesion molecule-3 (ICAM-3), leukointegrin adhesin, the platelet glycoprotein gp IIb/IIIa, cardiac myosin heavy chain, parathyroid hormone, rNAPc2 (which is an inhibitor of factor VIIa-tissue factor), MHC I, carcinoembryonic antigen (CEA), alpha-fetoprotein (AFP), tumor necrosis factor (TNF), CTLA-4 (which is a cytotoxic T lymphocyte-associated antigen), Fc-γ-1 receptor, HLA-DR 10 beta, HLA-DR antigen, L-selectin, IFN-γ, Respiratory Syncitial Virus, human immunodeficiency virus (HIV), hepatitis B virus (HBV), Streptococcus mutans, and Staphylococcus aureus.

Additional polypeptides specifically contemplated for purification according to the invention include recombinant fusion polypeptides comprising at least a portion of an Fc domain of an antibody. A polypeptide fused to an Fc domain and identical to or substantially similar to one of the following polypeptides is suitable for use in the present pharmaceutical composition: a flt3 ligand, a CD40 ligand, erythropoeitin, thrombopoeitin, calcitonin, Fas ligand, ligand for receptor activator of NF-kappa B (RANKL), tumor necrosis factor (TNF)-related apoptosis-inducing ligand (TRAIL), thymic stroma-derived lymphopoietin, granulocyte colony stimulating factor, granulocyte-macrophage colony stimulating factor, mast cell growth factor, stem cell growth factor, epidermal growth factor, RANTES, growth hormone, insulin, insulinotropin, insulin-like growth factors, parathyroid hormone, interferons, nerve growth factors, glucagon, interleukins 1 through 18, colony stimulating factors, lymphotoxin-β, tumor necrosis factor (TNF), leukemia inhibitory factor, oncostatin-M, and various ligands for cell surface molecules ELK and Hek (such as the ligands for eph-related kinases or LERKS).

Polypeptides suitable for purification according to the invention also include recombinant fusion polypeptides comprising an Fc domain of an antibody plus a receptor for any of the above-mentioned polypeptides or polypeptides substantially similar to such receptors. These receptors include: both forms of TNFR (referred to as p55 and p75), Interleukin-1 receptors (type 1 and 2), Interleukin-4 receptor, Interleukin-15 receptor, Interleukin-17 receptor, Interleukin-18 receptor, granulocyte-macrophage colony stimulating factor receptor, granulocyte colony stimulating factor receptor, receptors for oncostatin-M and leukemia inhibitory factor, receptor activator of NF-kappa B (RANK), receptors for TRAIL (TRAIL receptors 1, 2, 3, and 4), and receptors that comprise death domains, such as Fas or Apoptosis-Inducing Receptor (AIR).

Other suitable polypeptides include differentiation antigens (referred to as CD polypeptides) or their ligands or polypeptides substantially similar to either of these, which are fused to an Fc domain of an antibody. Such antigens are disclosed in Leukocyte Typing VI (Proceedings of the VIth International Workshop and Conference, Kishimoto, Kikutani et al., eds., Kobe, Japan, 1996). Similar CD polypeptides are disclosed in subsequent workshops. Examples of such antigens include CD27, CD30, CD39, CD40, and ligands thereto (CD27 ligand, CD30 ligand, etc.). Several of the CD antigens are members of the TNF receptor family, which also includes 41BB ligand and OX40. The ligands are often members of the TNF family, as are 41BB ligand and OX40 ligand. Accordingly, members of the TNF and TNFR families can be formulated according to the present invention.

Enzymatically active Fc fusion polypeptides or their ligands can also be purified according to the invention. Examples include recombinant fusion polypeptides comprising an Fc domain of an antibody fused to all or part of one of the following polypeptides or their ligands or a polypeptide substantially similar to one of these: metalloproteinase-disintegrin family members, various kinases, glucocerebrosidase, superoxide dismutase, tissue plasminogen activator, Factor VIII, Factor IX, apolipoprotein E, apolipoprotein A-I, globins, an IL-2 antagonist, alpha-1 antitrypsin, TNF-alpha Converting Enzyme, ligands for any of the above-mentioned enzymes, and numerous other enzymes and their ligands.

The methods of the invention can also be used for anti-idiotypic antibodies, or substantially similar polypeptides, including but not limited to anti-idiotypic antibodies against: an antibody targeted to the tumor antigen gp72; an antibody against the ganglioside GD3; or an antibody against the ganglioside GD2.

### Expression of Polypeptides

The Fc domain containing polypeptide of the present invention can be produced by living host cells that express the polypeptide, such as hybridomas in the case of antibodies, or host cells that that have been genetically engineered to produce the polypeptide in the case of fusion polypeptides or antibodies.

A wide variety of animal cell lines suitable for growth in culture are available from, for example, the American Type Culture Collection (ATCC, Manassas, Va.) and NRRL (Peoria, Ill.). Some of the more established cell lines used in industrial or academic laboratories and which are preferred are CHO, NSO, VERO, BHK, HeLa, Cos, CV1, MDCK, 293, 3T3, PC12, hybridoma, mycloma, and WI38 cell lines, to name but a few examples. The dihydrofolate reductase (DHFR)-deficient mutant cell lines (Urlaub et al., 1980, Proc Natl Acad Sci USA 77:4216 4220), DXB11 and DG-44, are the CHO host cell lines of choice because the efficient DHFR selectable and amplifiable gene expression system allows high level recombinant polypeptide expression in these cells (Kaufman R. J., 1990, Meth Enzymol 185:527 566). In addition, these cells are easy to manipulate as adherent or suspension cultures and exhibit relatively good genetic stability. In addition, new animal cell lines can be established using methods well known by those skilled in the art (e.g., by transformation, viral infection, and/or selection, etc.).

By in vitro cell culture is meant the growth and propagation of cells outside of a multicellular organism or tissue. Typically, in vitro cell culture is performed under sterile, controlled temperature and atmospheric conditions in culture plates (e.g., 10 cm plates, 96 well plates, etc.), or other adherent culture (e.g., on microcarrier beads) or in suspension culture in a reactor and/or in roller bottles. Cultures can be grown in shake flasks, small scale bioreactors, and/or large-scale bioreactors. A bioreactor is a device used to culture animal cells in which environmental conditions such as temperature, atmosphere, agitation, and/or pH can be monitored and adjusted. A number of companies (e.g., ABS Inc., Wilmington, Del.; Cell Trends, Inc., Middletown, Md.) as well as university and/or government-sponsored organizations (e.g., The Cell Culture Center, Minneapolis, Minn.) offer cell culture services on a contract basis.

Further, the methods and cell cultures of the invention (adherent or non-adherent and growing or growth arrested), can be small scale cultures, such as for example in 100 ml containers having about 30 ml of media, 250 ml containers having about 80 to 90 ml of media, 250 ml containers having about 150 to 200 ml of media. Alternatively, the cultures can be large scale such as for example 1000 ml containers having about 300 to 1000 ml of media, 3000 ml containers having about 500 to 3000 ml of media, 8000 ml containers having about 2000 to about 8000 ml of media, and 15000 ml containers having about 4000 ml to about 15000 ml of media. Both small scale and large scale culturing can be performed in bioreactors. In preferred embodiments, the size of the culture is at least about 100 liters, more preferably at least about 1000 liters, still more preferably at least about 5000 liters, even more preferably at least about 7000 liters.

Cell culture medium is defined, for purposes of the invention, as a medium suitable for growth of animal cells, and preferably mammalian cells, in in vitro cell culture. Typically, culture media contains a buffer, salts, energy source, amino acids, vitamins and trace essential elements. Any medium capable of supporting growth of the appropriate cell in culture can be used; as shown below by way of example, variations in a serum-free medium composition did not affect the superior results obtained when the high concentration media was fed to the cell culture.

Cell culture media suitable for use in the invention are commercially available. For example, any one or combination of the following media can be used: RPMI-1640 Medium, Dulbecco's Modified Eagle's Medium, Minimum Essential Medium Eagle, F-12K Medium, Iscove's Modified Dulbecco's Medium. When defined medium that is serum-free and/or peptone-free is used, the medium is usually enriched for certain amino acids and trace elements (see, for example, U.S. Pat. No. 5,122,469 to Mather et al., and U.S. Pat. No. 5,633,162 to Keen et al.). However, these enriched feeds are typically not more than 10× above the growth media.

When defined medium that is serum-free and/or peptone-free is used, the medium is usually enriched for particular amino acids, vitamins and/or trace elements (see, for example, U.S. Pat. No. 5,122,469 to Mather et al., and U.S. Pat. No. 5,633,162 to Keen et al.). Depending upon the requirements of the particular cell line used, medium also contains a serum additive such as Fetal Bovine Serum, or a serum replacement. Examples of serum-replacements (for serum-free growth of cells) are TCH™, TM-235™, and TCH™; these products are available commercially from Celox (St. Paul, Minn.).

In the methods and compositions of the invention, cells can be grown in serum-free, protein-free, growth factor-free, and/or peptone-free media. The term “serum-free” as applied to media includes any mammalian cell culture medium that does not contain serum, such as fetal bovine serum. The term “insulin-free” as applied to media includes any medium to which no exogenous insulin has been added. By exogenous is meant, in this context, other than that produced by the culturing of the cells themselves. The term “growth-factor free” as applied to media includes any medium to which no exogenous growth factor (e.g., insulin, IGF-1) has been added. The term “peptone-free” as applied to media includes any medium to which no exogenous protein hydrolysates have been added such as, for example, animal and/or plant protein hydrolysates.

Preferably, the medium used is serum-free, or essentially serum-free. By “essentially serum-free” is meant that less than about 2% serum is present, more preferably less than about 1% serum is present, still more preferably less than about 0.5% serum is present, yet still more preferably less than about 0.1% serum is present. By “serum free”, it is understood that the medium is preferably less than 0.1% serum and more preferably less than 0.01% serum.

The methods of the invention can be used in combination with other types of cell culture. For example, cell cultures can be serial subcultured in larger and larger volumes of culture medium to as to maintain the cells in exponential phase, and then converted to a batch culture system when a desired volume or cell density is achieved. Then, the batch cell culture can be fed using the methods of the invention. For example, a CHO cell culture can be grown and progressively transferred from a small scale culture to a large scale culture, and then seeded at a desired cell density into a batch cell culture. Once in the batch cell culture, the cells can be fed using the methods of the invention. CHO cells can be maintained in batch culture for as long as recombinant protein production occurs. Preferably, the batch culture is maintained in a production phase for about 2 to about 16 days, more preferably for about 6 to about 12 days.

Further, the methods of the invention can be used in combination with known or yet to be discovered methods of inducing the production of recombinant proteins. By “inducing conditions” is meant a technique to increase the relative production per cell of a desired recombinant protein during the production phase of the culture. Often, other cell processes (such as growth and division) are inhibited so as to direct most of the cells' energy into recombinant protein production. Such techniques include cold temperature shift, and additions of chemicals such as sodium butyrate (as described in U.S. Pat. No. 5,705,364 to Etcheverry et al., incorporated herein by reference), DMSO, DMF, DMA, TNF-alpha, phorbol 12-myristate 13-acetate, PMA, propionate, forskolin, dibutyryl cAMP, 2-aminopurine, adenine, adenosine, okadaic acid, and combinations of any of these techniques, to name just a few examples, as well as any yet to be described and/or discovered induction techniques. Typically, a batch culture of cells at high density is induced to produce the recombinant protein.

The invention can be used in the culture of cells that produce just about any protein, especially recombinant Fc domain containing proteins. Examples of useful expression vectors that can be used to produce proteins are disclosed in WO 01/27299, and the pDC409 vector described in McMahan et al., 1991, Embo J. 10:2821. A protein is generally understood to be a polypeptide of at least about 10 amino acids, more preferably at least about 25 amino acids, even more preferably at least about 75 amino acids, and most preferably at least about 100 amino acids.

Purification of Polypeptides

Polypeptides and proteins are purified from cell supernatants, cell extracts, tissue homogenates, or E. coli cytoplasm or inclusion bodies by various preparative precipitation, centrifugal, electrophoretic, filtration and chromatographic steps.

Salt precipitation, typically with ammonium sulfate, is a convenient and gentle first step and reduces the large volume of the starting material. Subsequent steps may use centrifugation, affinity purification on an antibody or substrate column or other affinity supports and a combination of size, charge, and hydrophobic chromatography. Final steps may include reverse phase HPLC. The desired protein is identified by a bioassay or enzyme activity, immunoassay and amino acid sequencing, and quality controlled by a variety of assays. Endotoxin is measured and removed if necessary.

The harvest process separates the antibody product released in the culture broth (conditioned medium) from the cells. Continuous centrifugation is commonly used to harvest large-scale cell culture broth ranging from 500 L to 20,000 L, resulting in a cell-free supernatant. Generation of a supernatant with low cell turbidity is desirable to limit clogging of filters downstream of the harvest process, thus, the supernatant can be further clarified through depth filtration followed by regular filtration.

Protein A chromatography is the most common purification step for antibodies and Fc domain containing proteins and polypeptides; it can purify the product to more than 98% purity and remove most process impurities, including proteases. Two major types of protein A resins are commercially available: Agarose-based resin from GE Healthcare and glass bead-based resin from Millipore. Both resins are robust enough to handle a high flux and have acceptable chemical resistance, including resistance to high concentration of urea, GuHC1, and reducing agents. Binding capacity can be in the range of 20 to 50 g per liter of resin, and both resins can be reused up to 200 times. Typical operating conditions of a protein A affinity chromatography step can be optimized in three key areas: resin type and sample loading residence time for optimal binding capacity; composition, pH, and volume of wash solution to minimize the HCP and rDNA levels in the pool; and composition and pH of elution solution to minimize turbidity, conductivity, dimer, and aggregate levels, as well as volume of the final pool, which is important for subsequent steps. Typical elution from protein A columns is by stepwise decrease in pH until the bound protein elutes. However, it has been found that the Fc domain proteins will often aggregate in a manner where they are still capable of binding to the column but are not a single protein. The present inventors have found that by using a gradient elution process, they have been able to separate the Fc domain containing protein aggregates typically found in the eluate from these columns. This suggests that the aggregates may have a different affinity for the column either lower because of the misshapen form they have assumed due to the aggregation, or possibly higher because the aggregates may have more than one Fc domain, which is the protein A binding portion of the protein.

Viral inactivation at low pH usually can follow protein A affinity chromatography and two representative viruses, specifically minute virus of mice (MVM) and murine leukemia virus (MuLV), are commonly used for process optimization. The protein A column pool is titrated with 10% acetic acid or 1M citric acid to a pH between 3.3 and 3.8 and incubated for 45 to 60 minutes at room temperature. Use of low pH for viral inactivation is effective for MuLV, whereas, MVM viral particles are not efficiently killed at low pH. After viral inactivation, the pH of the protein A pool is titrated up with 3M Tris buffer prior to the next step. Turbidity of the solution increases as the pH of the pool rises. The degree of turbidity of the solution after pH adjustment varies depending on process conditions for each product. In order to remove the turbidity, viral inactivation is followed by depth filtration or regular filtration. An optimized depth filtration step can greatly reduce turbidity, as well as host cell protein (HCP) and rDNA levels in the post-viral inactivation pool solution. In addition, depth filtration can efficiently remove MVM and MuLV viruses with log reduction values (LRV) of four and two, respectively.

A variety of chromatographic methods can be performed subsequent to protein A, and include size exclusion chromatography for native protein size, ion exchange, hydrophobic, reverse-phase based upon solubility separation, dye-binding and other chromatographic methods to characterize proteins.

Cation-exchange chromatography (CEX), hydrophobic-interaction chromatography (HIC), and anion-exchange chromatography (AEX), can be used for polishing. CEX chromatography used for bind-and-elution has proven to be a useful method to remove product-related impurities after protein A affinity chromatography. CEX resin screening criteria include less HCP binding, high product dynamic binding capacity at a relatively high conductivity, and high resolution to remove target protein variants. An ideal resin will leave about 70-80% of HCP and most of the DNA/RNA and endotoxin in the flow-through fraction.

Hydrophobic-interaction chromatography (HIC) is an efficient mode to remove dimers and aggregates in bind-and-elution fashion. However, this mode has relatively low yield and separation resolution for other product-related isomers, and has to contend with high salt concentration in the elution pools. Therefore, bind-and-elution through HIC is becoming less popular in antibody production. Instead, HIC chromatography in a flow-through mode is gaining interest as a way to remove a large percentage of aggregates. A slow flow rate is necessary because, like other protein binding during HIC chromatography, aggregate binding on HIC resin is residence time-dependent.

Ceramic hydroxyapatite (CHT) chromatography also can be a robust step to remove dimers and aggregates. Several technical issues regarding CHT need to be addressed, however, including lot-to-lot viability, extractables, and the halflife of the resin.

Several chromatography modes have proven very useful to remove trace amounts of impurities (e.g., DNA and endotoxin) and viruses. Among these, anion-exchange chromatography (AEX) is carried out using flow-through (FT) fashion, in which impurities bind to the resin and the product of interest flows through. However, packed-bed chromatography with FT-AEX requires columns with a very large diameter to permit high volumetric flow rates which are required to avoid a process bottleneck at the polishing step. In order to minimize the effect on the column operation by inadequate header design, a specific bed height is required for proper flow distribution. This leads to a large column volume, which is needed for fast flow but is not optimized for binding capacity. This disadvantage with AEX columns has led to the development of membrane chromatography or membrane adsorbers.

High Performance Liquid Chromatography (HPLC) is one of the most widely used analytical techniques. It utilizes a liquid mobile phase to separate the components of a mixture by forcing the components (analytes) dissolved in solvent to flow through a chromatographic column under high pressure. The mixture is resolved into its components based on the degree of interaction between the solute components and the stationary phase, defined as the immobile packing material in the column. HPLC instruments consist of a reservoir of mobile phase, a pump, an injector, a separation column, and a detector. The various components in the mixture pass through the column at different rates due to differences in their distribution between the mobile liquid phase and the stationary phase.

Ion exchange chromatography separates proteins or peptides based on charge characteristics. The net surface charge of a protein or peptide determines its adsorption to oppositely charged groups immobilized on the ion-exchange medium. Proteins are multivalent anions or cations, and the charge of a protein depends on the pH of the environment. When the pH is greater than the isoelectric point (pI) of the protein (number of positive charges equals the number of negative charges), the protein will have a net negative charge and will bind to an anion exchanger. When the pH is less than the isoelectric point, the protein will have a net positive charge and will bind to a cation exchanger. Once the sample is bound to the medium, unbound components are washed away and bound samples selectively eluted and collected.

Formulation of Pharmaceutical Compositions

To administer modified or selected polypeptides, including antibodies or Fc fusion polypeptides, of the invention to human or test animals, it is preferable to formulate the modified polypeptides in a composition comprising one or more pharmaceutically acceptable carriers. The phrase “pharmaceutically or pharmacologically acceptable” refer to molecular entities and compositions that do not produce allergic, or other adverse reactions when administered using routes well-known in the art, as described below. “Pharmaceutically acceptable carriers” include any and all clinically useful solvents, dispersion media, coatings, antibacterial and antifungal agents, isotonic and absorption delaying agents and the like. Examples include, but are not limited to, any of a number of standard pharmaceutical carriers such as sterile phosphate buffered saline solutions, bacteriostatic water, and the like. A variety of aqueous carriers may be used, e.g., water, buffered water, 0.4% saline, 0.3% glycine and the like, and may include other proteins for enhanced stability, such as albumin, lipoprotein, globulin, etc., subjected to mild chemical modifications or the like.

Exemplary polypeptide concentrations in the formulation may range from about 0.1 mg/ml to about 180 mg/ml or from about 1 mg/mL to about 100 mg/mL, or from about 5 mg/mL to about 50 mg/mL, or alternatively from about 20 mg/mL to about 40 mg/mL. An aqueous formulation of the polypeptide may be prepared in a pH-buffered solution, for example, at pH ranging from about 4.5 to about 6.5, or from about 4.8 to about 5.5, or alternatively about 5.0. Examples of buffers that are suitable for a pH within this range include acetate (e.g. sodium acetate), succinate (such as sodium succinate), gluconate, histidine, citrate and other organic acid buffers. The buffer concentration can be from about 1 mM to about 200 mM, or from about 10 mM to about 60 mM, depending, for example, on the buffer and the desired isotonicity of the formulation.

A tonicity agent, which may also stabilize the polypeptide, may be included in the formulation. Exemplary tonicity agents include polyols, such as mannitol, sucrose or trehalose. Preferably the aqueous formulation is isotonic, although hypertonic or hypotonic solutions may be suitable. Exemplary concentrations of the polyol in the formulation may range from about 1% to about 15% w/v.

A surfactant may also be added to the polypeptide formulation to reduce aggregation of the formulated polypeptide and/or minimize the formation of particulates in the formulation and/or reduce adsorption. Exemplary surfactants include nonionic surfactants such as polysorbates (e.g. polysorbate 20, or polysorbate 80) or poloxamers (e.g. poloxamer 188). Exemplary concentrations of surfactant may range from about 0.001% to about 0.5%, or from about 0.005% to about 0.2%, or alternatively from about 0.004% to about 0.01% w/v.

In one embodiment, the formulation contains the above-identified agents (i.e. polypeptide , buffer, polyol and surfactant) and is essentially free of one or more preservatives, such as benzyl alcohol, phenol, m-cresol, chlorobutanol and benzethonium Cl. In another embodiment, a preservative may be included in the formulation.

Therapeutic formulations of the polypeptide are prepared for storage by mixing the polypeptide having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives; low molecular weight (less than about 10 residues) polypeptides; proteins, such as serum albumin, gelatin, or immunoglobulins; hydrophilic polymers such as polyvinylpyrrolidone; amino acids; monosaccharides, disaccharides, and other carbohydrates; chelating agents such as EDTA; salt-forming counter-ions such as sodium; metal complexes; and/or non-ionic surfactants.

Other exemplary descriptions of pharmaceutical formulations for antibodies may be found in US 2003/0113316 and U.S. Pat. No. 6,171,586, each incorporated herein by reference in its entirety.

The formulations to be used for in vivo administration must be sterile. The compositions of the invention may be sterilized by conventional, well known sterilization techniques. For example, sterilization is readily accomplished by filtration through sterile filtration membranes. The resulting solutions may be packaged for use or filtered under aseptic conditions and lyophilized, the lyophilized preparation being combined with a sterile solution prior to administration.

For injection, the pharmaceutical formulation and/or medicament may be a powder suitable for reconstitution with an appropriate solution as described above. Examples of these include, but are not limited to, freeze dried, rotary dried or spray dried powders, amorphous powders, granules, precipitates, or particulates. For injection, the formulations may optionally contain stabilizers, pH modifiers, surfactants, bioavailability modifiers and combinations of these.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant invention.

The polypeptide may be administered by any suitable means, including parenteral, subcutaneous, intraperitoneal, intrapulmonary, and intranasal, and, if desired for local treatment, intralesional administration. Parenteral infusions include intravenous, intraarterial, intraperitoneal, intramuscular, intradermal or subcutaneous administration. In addition, the polypeptide is suitably administered by pulse infusion, particularly with declining doses of the polypeptide. The polypeptides of the invention can be administered intravenously in a physiological solution at a dose ranging between 0.01 mg/kg to 100 mg/kg at a frequency ranging from daily to weekly to monthly (e.g. every day, every other day, every third day, or 2, 3, 4, 5, or 6 times per week), preferably a dose ranging from 0.1 to 45 mg/kg, 0.1 to 15 mg/kg or 0.1 to 10 mg/kg at a frequency of 2 or 3 times per week, or up to 45 mg/kg once a month.

The formulations may be stored in a prefilled syringe or vial and may be part of a kit.

In addition to formulation for pharmaceutical compounds, the polypeptides of the invention may be formulated in a photo-oxidation-protective environment. For example, the polypeptide may be formulated and stored, or in a dark or opaque container that is impervious to oxidative light. Further, the polypeptide may be stored in an oxygen free environment comprising stable, non-oxidizing gases, such as nitrogen, helium, argon and neon.

Also contemplated is formulation of the polypeptide of the invention in solution with oxidation-protective agents, including but not limited to free radical scavengers, such as mannitol, methionine, histidine, casein, ascorbic acid, and N-acetylcysteine.

Additional aspects and details of the invention will be apparent from the following examples, which are intended to be illustrative rather than limiting.

### EXAMPLES

### Materials.

All antibodies are reference standards unless specified. Tert-buytl hydrogen peroxide (TBHP) solution (70%) was purchased from Alfa Aesar. All other reagents were purchased from Sigma (St. Louis, Mo.) unless specified.

### pH Gradient Protein A Chromatography.

POROS A/20 Protein A columns were purchased from Applied Biosystems. The chromatographic system is an Agilent 1100 HPLC equipped with a diode-array detector, autosampler, micro-flow cell (Agilent, Palo Alto, Calif.). UV absorbance was monitored at 280 nm. The buffers were (A) 20 mM Tris, 150 mM NaCl, pH 7.0 and (B) 20 mM Acetate, 150 mM NaCl, pH 3.1. Fifty micrograms of antibody was injected to the Protein A column after it was equilibrated at 0% B for at least 20 min.

The column was kept at room temperature. The pump gradient is described in Table 1.

TABLE 1
The pump gradient of pH gradient Protein A chromatography

| Time (min) | A (%) | B (%) | Flow Rate (mL/min) |
|---|---|---|---|
| 0.0 | 100 | 0 | 1.0 |
| 2.1 | 100 | 0 | 1.0 |
| 22.1 | 65 | 35 | 1.0 |
| 23.0 | 0 | 100 | 1.0 |
| 25.5 | 0 | 100 | 1.0 |
| 25.6 | 100 | 0 | 1.0 |
| 30.0 | 100 | 0 | 1.0 |

### Forced Oxidation by TBHP.

The original TBHP solution (˜70%) was diluted to 10% with 10 mM sodium acetate buffer (pH 5.2). An aliquot of 10% TBHP solution was added to the antibody solution (70 mg/ml) with volume ratio 1:9, so the final TBHP concentration is 1%. The mixture was incubated at 37° C. After various incubation times, a fraction was taken out and diluted into 100 folds of ice-cold acetate buffer. The solution was quickly buffer exchanged three times with the same acetate buffer using Vivaspin 20 with membrane cut-off at 10,000 MW.

### Lys-C Enzymatic Digestion.

The concentration of antibody samples were adjusted to 30 mg/ml, then diluted to 13 mg/ml with 8 M guanidine hydrochloride, 0.5 M Na₂HPO4, 0.5 M NaH₂PO4, 20 mM NEM to a total volume of 100 ul. The solution was then placed at 37° C. for 2 h to denature the antibody. Lys-C digestion was performed for 24 hours at 37° C. by mixing 15 ul denatured antibody, 85 ul digestion buffer (8 M urea, 0.2 M phosphate, 40 mM NH₂OH, pH 7.0), 90 ul H₂O and 10 ug Lys-C (Waco Chemicals). The samples were quenched with 15 ul of 5% TFA and later stored at −70° C.

### LC/MS of Lys-C Digests.

The Lys-C digests were separated on a Jupiter C18 column (250×2.0 mm, Phenomex, Calif.) using a two step linear gradient from 2 to 22% B over 38 min and then from 22% to 42% over 80 min. Solvent A was 0.1% TFA in water, and solvent B was 0.1% TFA, 90% acetonitrile and 10% water. The column temperature was maintained at 50 C. Before the sample injection, the column was equilibrated with 0% solvent B for 2 hours. The flow rate was 0.2 ml/min, and a total of 50 ug of protein digest was injected onto the column for analysis. An Agilent 1100 HPLC was equipped with a diode-array detector, autosampler and binary pumps was used to separate peptides. The UV wavelength was set at 215 nm. The HPLC was directly coupled to a Finnigan LCQ Deca ion trap mass spectrometer equipped with an electrospray ionization source.

### Results and Discussion

Protein A, a 42 kDa membrane protein in the cell wall of Staphylococcus aureus, specifically binds to the Fc domain of immunoglobulin G (IgG) from various mammalian species. Protein A affinity chromatography has been well established in the large scale purification of antibody or Fc fusion proteins. The wide use of Protein A chromatography for purification purposes is due to its high specificity. In typical bind and release mode, Protein A tightly binds the Fc portion of antibody under neutral pH while non-product related impurities being washed away, then the acidic pH buffer is quickly introduced to release the antibody from the Protein A column.

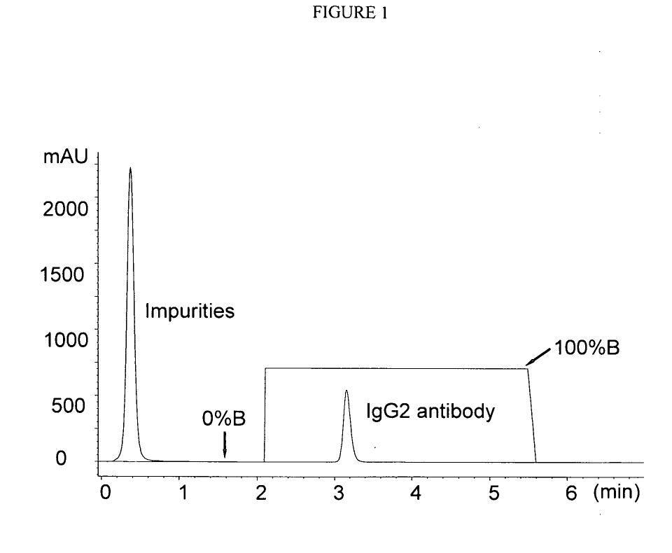

The typical chromatogram of a crude IgG2 antibody cell culture sample under bind and release mode is shown in FIG. 1. A sharp antibody peak was observed and the non-product impurities such as lipid, DNA and host cell proteins, were eluted at the beginning.

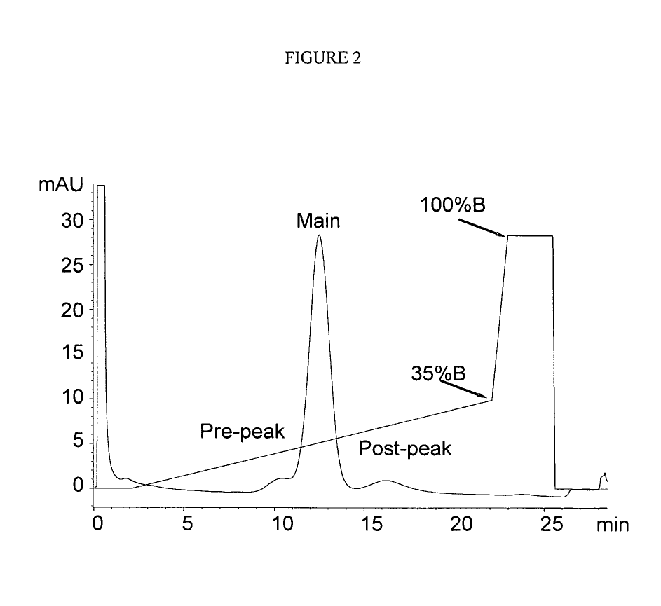

In this embodiment, a pH gradient is generated by mixing a neutral pH buffer and an acidic pH buffer to elute the column bound IgGs. A pre-peak and a post-peak were observed in the chromatogram of the same sample in FIG. 1 under this mode of separation (FIG. 2), indicating that pH gradient not only can separate non-product impurities, it can also separate product related impurities.

### Characterization of the Post-Peak in pH Gradient Protein A Chromatography

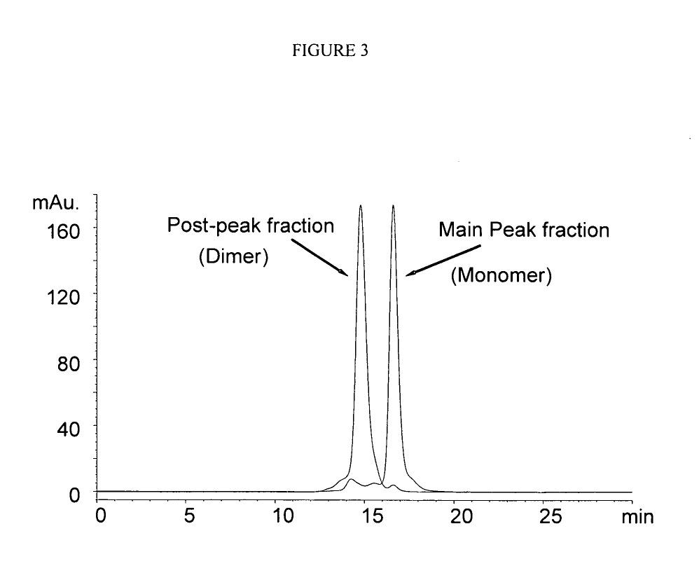

To extensively characterize the post-peak species, the postpeak and main peak fractions of antibody were collected and concentrated. These two fractions were analyzed by Size Exclusion Chromatography (FIG. 3). The main peak species collected from pH gradient Protein A chromatography is shown to be monomer and the post-peak species is dimer (aggregate). This result indicates that the pH gradient Protein A chromatography can separate monomer and aggregates. One potential mechanism to explain this separation, while not intending to be limiting, is due to aggregates having more than one Fc and thus a stronger interaction with protein A.

### Characterization of the Pre-Peak in pH Gradient Protein A Chromatography

To extensively characterize the pre-peak species, the prepeak and the main peak fractions of antibody were collected, concentrated and buffer exchanged to 10 mM acetate buffer, pH 5.2. SEC (size exclusion chromatography) analysis shows that pre-peak and the main peak are both monomers. The peptide map analysis was applied to the pre-peak and the main peak fractions to find out the possible modifications. Significant oxidation at two methionine sites was observed in the pre-peak fraction. The percentage of methionine oxidation was quantified by peptide mapping and results are shown in Table 2. Met 252 and Met 428 have higher oxidation levels in the pre-peak fraction than the main peak fraction with 46.5% and 30% oxidation level respectively.

TABLE 2
Percentage of methionine oxidation in pre-peak and main peak
fractions collected from pH gradient Protein A chromatography.

| | % Oxidized in Met 252 | % Oxidized in Met 428 |
|---|---|---|
| Pre-peak fraction | 46.5 | 30.0 |
| Main Peak fraction | 2.9 | 4.6 |

Both methionines are located in Fc and structural analysis show that these two methionines interact with Protein A. The binding sites between Fc and Protein A involve dozens of residues in Fc. Thus, variants with modifications in these residues potentially can also be detected by this approach.

### Removal of Aggregates and Fc Modified Form

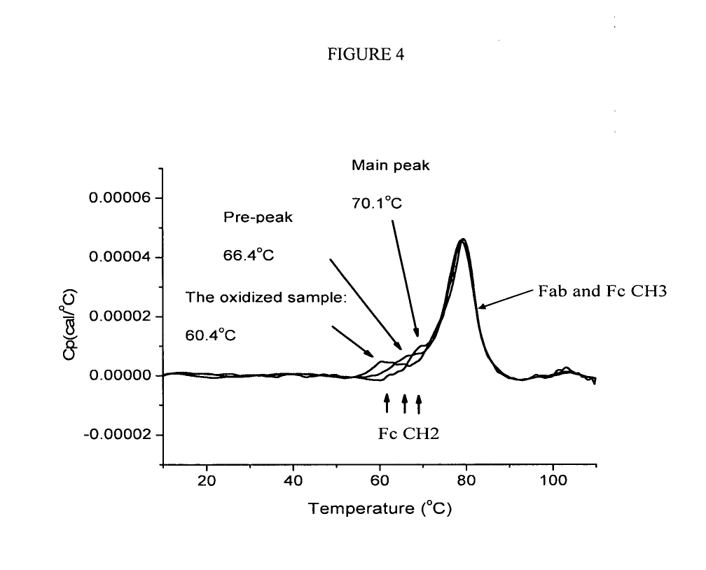

Differential scanning calorimetry (DSC) was used to test the stability of the Fc oxidized antibody. Three samples were tested and the main peak fractions collected for the pH gradient Protein A chromatography, the pre-peak fraction and the fully oxidized antibody were treated by oxidized reagent, TBHP. The percentage of methionine oxidation in fully oxidized antibody is close to 100%. The DSC scans of three samples in 10 mM sodium acetate buffer at pH 5.2 are shown in FIG. 4. Two major endothermic thermal transitions are observed for the samples. The first thermal transition is from the unfolding of the Fc CH2 domain and the second one is from the unfolding of the Fab and Fc CH3 domains.

The profiles of the Fab and CH3 domains are identical for all of the samples. This suggests that there is no change for the Fab and CH3 domains among the samples. The profiles of the CH2 domain changes significantly. The thermal transition temperatures of the CH2 domain for the main peak, pre-peak, and fully oxidized sample are 70.1±0.5, 66.4±0.5, and 60.4±0.5° C., respectively.

In summary, the pH gradient Protein A chromatography can remove aggregates and Fc modified form such as oxidized form. Both of them will lower the quality of the antibody therapeutics.

### Antibody Isotypes and Fc Fusion Protein

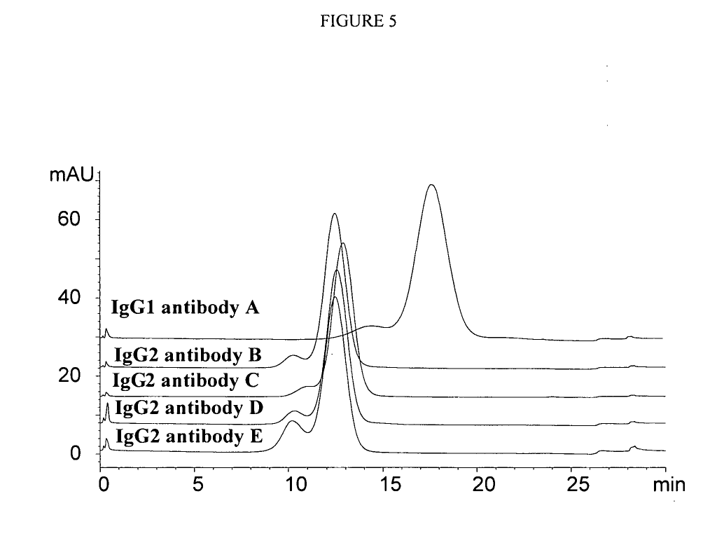

pH gradient Protein A chromatography has been applied to several isotypes of antibodies (FIG. 5). IgG2 antibodies have similar retention times and all have the pre-peak. IgG1 antibody elutes about 5 min later probably because it has slightly different Fc sequence. Interestingly, a pre-peak can also be observed and proved to be as Fc oxidized form as well. All antibodies tested here are final purified bulk and have very small amount of aggregates (<1%). The post-peaks for them are not obvious.

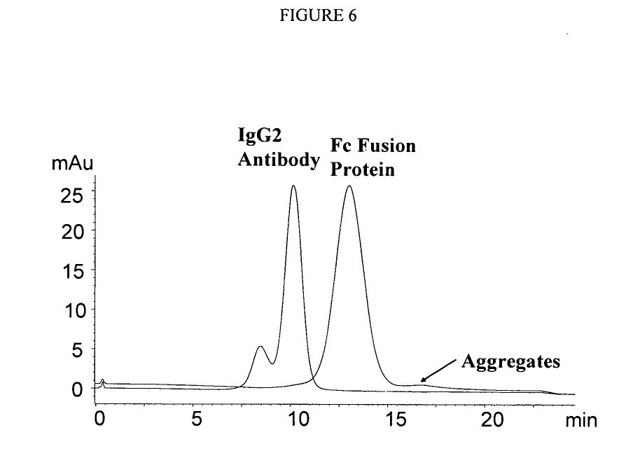

The pH gradient protein A chromatogram for an Fc fusion protein reference standard is shown in FIG. 6. Post-peak (aggregates) can be separated.

The sequence of IgG4 Fc is very similar to IgG1 Fc sequence, so this method potentially can also be applied to IgG4, another major IgG class being used or investigated for human therapeutic applications. Human IgG3, rarely used in therapeutic applications, doesn't bind to the Protein A. All these results indicate this approach can be widely used for the purification of wide array of therapeutic antibodies and Fc fusion proteins.

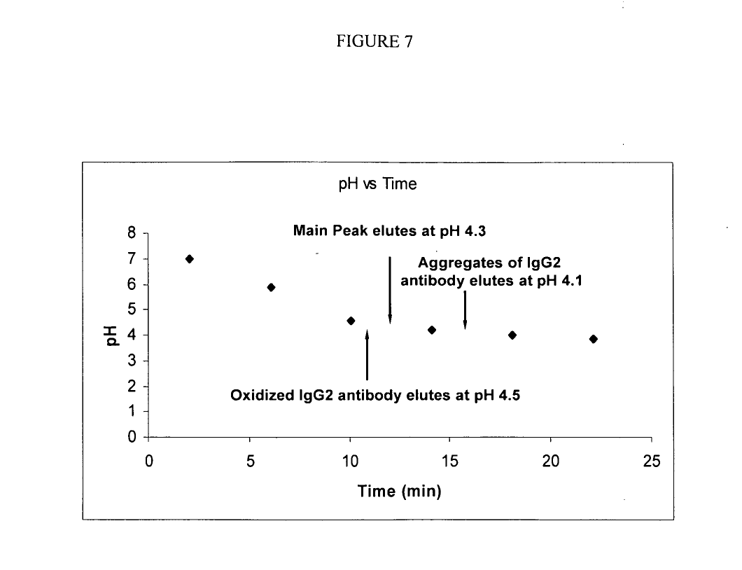

The pH during the gradient was measured offline and shown in FIG. 7. The main peak of a IgG2 antibody elutes at pH 4.3. The oxidized and aggregate forms elute at pH 4.1 and pH 4.3. The IgG1 elutes at slightly lower pH.

Numerous modifications and variations in the invention as set forth in the above illustrative examples are expected to occur to those skilled in the art. Consequently only such limitations as appear in the appended claims should be placed on the invention.

What is claimed:

1. A method of purifying a human Fc domain containing polypeptide comprising binding said polypeptide to protein A and eluting with a pH gradient.

2. The method of claim 1, wherein the top of the pH gradient starts above pH 7.0 and bottom of the gradient is at or below pH 4.0 and at or above pH 3.0.

3. The method of claim 2, wherein the top of the gradient is at or near pH 6.0.

4. The method of claim 1, wherein the gradient starts near pH 6 and ends at about pH 4.

5. The method of claim 1, wherein the Fc domain containing polypeptide is an antibody.

6. The method of claim 1, wherein the Fc domain containing polypeptide is a fusion protein comprising an antibody Fc domain fused to a heterologous protein sequence.

7. The method of claim 1, wherein the purification is part of a manufacturing scale process.

8. The product purified by a method comprising purifying an Fc domain containing polypeptide comprising binding said polypeptide to protein A and eluting with a pH gradient.

9. The method of claim 8, wherein the top of the pH gradient starts above pH 7.0 and bottom of the gradient is at or below pH 4.0 and at or above pH 3.0.

10. The method of claim 9, wherein the top of the gradient is at or near pH 6.0.

11. The method of claim 8, wherein the gradient starts near pH 6 and ends at about pH 4.

12. The product of claim 8 wherein the Fc domain containing polypeptide is an antibody.

13. The product of claim 13, wherein the antibody does not have oxidized methionines in the Fc domain.
